(19) United States
(12) Patent Application Publication
(10) Pub. No.: US 2011/0236386 A1
(43) Pub. Date: Sep. 29, 2011

(54) NOVEL METHOD FOR PREDICTION OF CARDIOVASCULAR DISEASE RISK IN TYPE 2 DIABETES

(76) Inventor: Mark B. Zimering, Lyons, NJ (US)

(21) Appl. No.: 13/041,323

(22) Filed: Mar. 4, 2011

Related U.S. Application Data
(60) Provisional application No. 61/310,548, filed on Mar. 4, 2010.

(51) Int. Cl. A61K 39/395; G01N 33/566; G01N 21/75; G01N 23/00; A61P 9/10; A61P 9/00

(52) U.S. Cl. 424/135.1; 435/7.92; 436/501; 424/158.1; 424/145.1; 424/133.1; 435/7.9

(57) ABSTRACT

The invention provides a method for diagnosing an increased risk of cardiovascular disease (CVD, including coronary heart disease (CHD)) in patients with type 2 diabetes comprising detecting basic fibroblast growth factor (bFGF) in a sample from the patient, an increased level of bFGF being indicative of increased risk of CHD, thereby diagnosing an increased risk of CHD in diabetic patients.

This patent application claims the benefit of the filing dates of U.S. Ser. No. 61/310,548, filed Mar. 4, 2010, the contents of all of which are herein incorporated by reference in their entireties into the present patent application.

Throughout this application various publications are referenced. The disclosures of these publications in their entireties are hereby incorporated by reference into this application in order to more fully describe the state of the art to which this invention pertains.

### FIELD OF THE INVENTION

The invention relates to methods for diagnosing or monitoring pathological complications in subject with type 2 diabetes, such as cardiovascular disease (CVD, including, e.g., coronary heart disease (CHD)) and coronary artery diseases.

### BACKGROUND OF THE INVENTION

Coronary heart disease is the leading cause of death in adults in the United States (1), and adults with type 2 diabetes suffer a disproportionately high rate of coronary heart disease morbidity and mortality (2). Dyslipidemia, hypertension, pro-thrombotic and pro-inflammatory factors each contribute to accelerated atherosclerosis in obese type 2 diabetes (3). Still the pathophysiologic mechanism(s) accounting for a substantially increased risk for coronary heart disease in adult type 2 diabetes remain unclear.

A possible role for hyperglycemia as a mediator of cardiovascular disease risk was recently explored in the Veterans Affairs Diabetes Trial (VADT). Seventeen hundred ninety one older adults with advanced type 2 diabetes were randomized to standard (STD) or intensive (INT) glycemic treatment groups in which blood pressure and lipids were maintained at similar, desirable levels (4). After an average of six years' treatment, cardiovascular disease outcomes did not differ significantly according to original glycemic treatment group assignment (5). The current report is from a planned secondary analysis to the VADT which investigated whether plasma basic fibroblast growth factor, an angiogenic growth factor implicated in early atherosclerosis, may predict the occurrence of coronary heart disease in adult type 2 diabetes.

Basic fibroblast growth factor (bFGF) is a potent mitogen in endothelial cells and smooth muscle cells which is released following endothelial injury (6), and is capable of inducing smooth muscle cell migration and proliferation important in neointimal formation (7, 8). Plasma basic FGF is low or undetectable in healthy subjects (9), but increases in micro-albuminuric adult type 2 diabetes (10) and in coronary artery disease (11). Micro- or macroalbuminuria are markers of diffuse endothelial damage (12) associated with increased cardiovascular disease mortality in adult diabetes (13). However, there has been no prior report of plasma bFGF itself as a possible marker for cardiovascular or coronary heart disease risk in adult type 2 diabetes.

Applicants herein report the first evidence that substantially increased plasma bFGF-IR is associated with an increased risk for coronary heart disease morbidity and mortality in middle-aged or older adults with advanced type 2 diabetes in a subset of 399 patients undergoing VADT treatment for an average of six years.

### SUMMARY OF THE INVENTION

The invention provides a method for diagnosing an increased risk of cardiovascular disease (CVD) in subjects (e.g., diabetic patients, including advanced type 2 diabetic patients) having elevated levels of basic fibroblast growth factor, by contacting a sample from the patient with an agent (e.g. a detectable agent) that is able to detect basic fibroblast growth factor (bFGF and/or its levels) in the sample.

The invention further provides a method for monitoring the course of a pathological complication in a subject (e.g., diabetic patients, including advanced type 2 diabetic patients), which comprises quantitatively determining in a first sample from the subject, the level of bFGF, then comparing the amount so determined with the amount present in a second sample from the subject, such samples being taken at different points in time, a difference in the amounts determined, being indicative of the course of a pathological complication: an increase in amount of bFGF indicating increased risk of cardiovascular disease, and a decrease in the amount indicating reduced risk of cardiovascular disease.

The invention further provides a method for treating cardiovascular disease including coronary heart disease (CHD) in subjects (e.g., diabetic patients, including advanced type 2 diabetic patients) comprising administering to the subject an effective amount of an agent (e.g. an antibody or fragment thereof) that recognizes and binds basic fibroblast growth factor (bFGF) in a subject, thereby treating cardiovascular disease including coronary heart disease (CHD).

### BRIEF DESCRIPTION OF THE DRAWINGS

FIG. 1 shows occurrence of coronary heart disease (CHD) events by bFGF level. The difference in time to occurrence of first CHD event for bFGF groups was statistically significant, p=0.03. Dashed line indicates group with plasma bFGF>50 pg/mL; solid line 0-50 pg/mL. See Example 1 below.

FIG. 2. Hazard ratio for cardiovascular disease (CVD) events by baseline known diabetes duration. Triangles indicated point estimates and bars denote 95% confidence intervals. Point estimates were obtained from the multivariate adjusted model that included age, prior CV event, baseline bFGF, treatment, duration and treatment×duration and are illustrated for 5-year intervals between 0-30 years' baseline duration.

### DETAILED DESCRIPTION OF THE INVENTION

### Definitions

All scientific and technical terms used in this application have meanings commonly used in the art unless otherwise specified. As used in this application, the following words or phrases have the meanings specified.

As used herein, an “agent” refers to a compound capable of forming a complex with the basic fibroblast growth factor (bFGF) in a sample. For example, the agent can be an antibody. The antibody may be labeled for detection.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they bind bFGF.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)).

“Antibody fragments” comprise a portion of an intact antibody, preferably comprising the antigen binding region thereof. Examples of antibody fragments include Fab, Fab′, F(ab′)₍₂₎, and Fv fragments; diabodies; linear antibodies; single-chain antibody molecules; and multispecific antibodies formed from antibody fragments. The monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol, Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used herein, a “subject” or a “patient” is used interchangeably and refers to any mammal. For example a subject can be, but is not limited to, a human, mouse, rat, pig, monkey and ape, cow, sheep and horse.

As used herein “individual,” “subject,” or “patient” is a vertebrate. In certain embodiments, the vertebrate is a mammal. Mammals include, but are not limited to, farm animals (such as cows and pigs), sport animals, pets (such as cats, dogs, and horses), primates, mice and rats. In certain embodiments, a mammal is a human.

As used herein, a “sample” refers to a biological sample from a subject. For example, the sample can be a fluid (e.g., urine, whole blood, serum or plasma, seminal, saliva, tears or other fluid), a cell or tissue from a subject.

As used herein, a “label” or “detectable marker” refers to an indicator that can be attached to an agent and detected. Examples of labels or detectable markers include, but are not limited to radiolabels, enzymes, chromophores, fluorescent compounds, chemiluminescent dyes, phosphorescent, latex and magnetic particles; dye crystalites, gold, silver, and selenium colloidal particles; metal chelates; coenzymes; electroactive groups; oligonucleotides, and stable radicals.

Cardiovascular disease (CVD) includes but is not limited to coronary heart disease (CHD), coronary artery disease, ischemic coronary artery disease, congestive heart failure, cerebrovascular disease, peripheral arterial disease, myocardial infarction, heart failure, stroke, amputation, cardiovascular death, coronary revascularization, cerebrovascular revascularization, peripheral revascularization or inoperable coronary artery disease.

Coronary heart disease (CHD) includes but is not limited to myocardial infarction, coronary revascularization, inoperable coronary artery disease or cardiovascular death.

### METHODS OF THE INVENTION

The present invention provides methods for diagnosing an increased risk of cardiovascular disease (CVD, including coronary heart disease (CHD)) in subjects (e.g., diabetic patients, including advanced type 2 diabetic patients). The method comprises detecting the level of basic fibroblast growth factor (bFGF) in a sample from the patient. An increase in the level of bFGF is indicative of increased risk of CVD.

In one embodiment, the detection step comprises obtaining a biological sample from a subject and contacting said sample with an agent capable of forming a detectable complex with bFGF in the sample. The complex may be subsequently detected. Detection includes any means of detecting, including direct and indirect detection. The agent can be labeled so as to produce a detectable signal with a compound such as a radiolabel, an enzyme, a chromophore and a fluorescer, chemiluminescent dyes, phosphorescent, latex and magnetic particles; dye crystalites, gold, silver, and selenium colloidal particles; metal chelates; coenzymes; electoactive groups; oligonucleotides, and stable radicals.

The invention also provides methods of detecting and quantitatively determining the concentration of bFGF in a biological fluid sample. For example, in one embodiment, the detection step comprises obtaining a biological sample from a subject and contacting said sample with an agent (e.g., an antibody or fragment thereof) that recognizes and binds bFGF. The antibody may be bound to a detectable marker before or after binding bFGF. The term “antibody” is used in the broadest sense and specifically covers monoclonal antibodies (including full length monoclonal antibodies), polyclonal antibodies, multispecific antibodies (e.g., bispecific antibodies), and antibody fragments so long as they bind bFGF. In a further embodiment, the method comprises contacting the agent so bound to bFGF with a second agent (e.g. an antibody or fragment thereof) labeled with a detectable marker so as to form a complex. The complex comprises the agent bound to bFGF and optionally, a second agent. The complex (or a component thereof) may be subsequently detected and optionally quantified.

In yet another embodiment, the method comprises contacting a solid support with an excess of one or more antibodies which forms (preferably specifically forms) a complex with bFGF under conditions permitting the monoclonal antibody to attach to the surface of the solid support. The resulting solid support to which the antibody is attached is then contacted with a biological fluid sample so that the bFGF in the biological fluid binds to the antibody and forms a bFGF-antibody complex. The complex can be labeled directly or indirectly with a detectable marker. Alternatively, either the bFGF or the antibody can be labeled before the formation the complex. The complex can then be detected and quantitatively determined thereby detecting and quantitatively determining the concentration of bFGF in the biological fluid sample. A high concentration of bFGF in the sample relative to normal cells being indicative of an increased risk of CVD.

In an embodiment of the invention, the agent which forms a complex with bFGF is an antibody or a fragment thereof that recognizes and binds bFGF. The anti-bFGF antibody or the fragment thereof may be labeled with a detectable marker such as a radiolabel, an enzyme, a chromophore and a fluorescer. In a further embodiment of the invention, the antibody may be a polyclonal antibody, a monoclonal antibody, a chimeric antibody, a humanized antibody or a fully-human antibody. In an additional embodiment of the invention, the antibody fragment is selected from the group consisting of Fv fragments, F(ab′) fragments, single chain antibodies and F(ab′)₂ fragments.

The most preferred antibodies will selectively bind to bFGF and will not bind (or will bind weakly) to non-bFGF proteins. The most preferred antibodies will specifically bind to bFGF. It is intended that the term “specifically bind” means that the antibody predominantly binds to bFGF. Anti-bFGF antibodies that are particularly contemplated include monoclonal and polyclonal antibodies as well as fragments thereof (e.g., recombinant proteins) containing the antigen binding domain and/or one or more complement determining regions of these antibodies. These antibodies can be from any source, e.g., rat, dog, cat, pig, horse, mouse or human.

In an embodiment of the invention, the detection comprises quantitatively determining the amount of above said complex. The measurement can be a direct measurement of the labeled agent(s)-bFGF complex.

In an embodiment of indirect measurement, a predetermined amount of labeled agent is allowed to form a complex with bFGF in the sample. The complex so formed is subsequently removed by any of several means for example by affinity chromatography. The residual labeled agent left behind after removal of the complex may be quantitatively measured. The measurement may be inversely proportional to the concentration of bFGF. Use of immunologically reactive fragments, such as the Fab, Fab′, or F(ab′)₂ fragments is often preferable, especially in a therapeutic context, as these fragments are generally less immunogenic than the whole immunoglobulin.

In another embodiment, the invention provides various immunological assays useful for the detection of bFGF protein. Such assays may comprise one or more bFGF antibodies capable of recognizing and binding a bFGF protein, and include various immunological assay formats well known in the art, including but not limited to various types of precipitation, agglutination, complement fixation, radioimmunoassays (RIA), enzyme-linked immunosorbent assays (ELISA), enzyme-linked immunofluorescent assays (ELIFA) (H. Liu et al. Cancer Research 58: 4055-4060 (1998)), immunohistochemical analysis and the like.

In particular embodiments of the invention, the level of bFGF that is indicative of increased risk of cardiovascular disease including coronary heart disease in subjects (e.g., diabetic and/or cancer patients, including advanced type 2 diabetic patients) includes but is not limited to: greater than 15 pg/mL; greater than 20 pg/mL; greater than 25 pg/mL; greater than 30 pg/mL; greater than 35 pg/mL; greater than 40 pg/mL; greater than 45 pg/mL; greater than 50 pg/mL; greater than 55 pg/mL; greater than 60 pg/mL; greater than 65 pg/mL; greater than 70 pg/mL; greater than 75 pg/mL; greater than 80 pg/mL; greater than 85 pg/mL; greater than 90 pg/mL; greater than 95 pg/mL; greater than 100 pg/mL; greater than 105 pg/mL; greater than 110 pg/mL; greater than 115 pg/mL; greater than 120 pg/mL; greater than 125 pg/mL; greater than 130 pg/mL; greater than 135 pg/mL; greater than 140 pg/mL; greater than 145 pg/mL; greater than 150 pg/mL; greater than 155 pg/mL; greater than 160 pg/mL; greater than 165 pg/mL; greater than 170 pg/mL; greater than 175 pg/mL; greater than 180 pg/mL; greater than 185 pg/mL; greater than 190 pg/mL; greater than 195 pg/mL or greater than 200 pg/mL.

In one embodiment of the invention, sample obtained from the subject is a biological fluid. The biological fluid may be urine, blood serum or plasma.

The invention further provides a method for monitoring the course of a pathological complication in a subject (e.g., diabetic and/or cancer patients, including advanced type 2 diabetic patients). The method comprises quantitatively determining the presence of basic fibroblast growth factor (bFGF) in a first biological sample from the subject using the detection methods discussed above. The amount of bFGF so determined is compared with the amount of bFGF present in a second biological sample from the subject, also determined using the detection methods discussed above. The two samples are taken at different points in time. A difference in the amounts of basic fibroblast growth factor (bFGF) between the first and second sample is indicative of the course of the pathological condition. In one embodiment, an increase in the amount of bFGF is indicative of increased risk of coronary heart disease.

In one embodiment, the pathological complication is cardiovascular disease (CVD). Cardiovascular disease (CVD) includes but is not limited to coronary heart disease (CHD), myocardial infarction, coronary revascularization, inoperable coronary artery disease and cardiovascular death.

In an embodiment of the invention, type 2 diabetes is advanced type 2 diabetes. In accordance with the practice of the invention, advanced type 2 diabetes generally includes patients that have had diabetes on average for 11 years, on average their diabetes was not well-controlled, and for these reasons they tended to have a fairly significant prevalence of microvascular complications.

In another embodiment of the invention, diabetes patients (e.g. type 2 diabetes patients) can be monitored for bFGF levels following intensive treatment. Increased bFGF levels as compared to their baseline levels (pretreatment) indicative of risk for CVD. Risk calculations based on years with type 2 diabetes.

The invention further provides a method for treating cardiovascular disease (CVD) in a subject. The method comprises administering to the subject an effective amount of an antibody that recognizes and binds basic fibroblast growth factor (bFGF), thereby treating cardiovascular disease (CVD).

In one embodiment, unconjugated bFGF antibody (including monoclonal, polyclonal, chimeric, humanized, fully human and fragments thereof (e.g., recombinant proteins)) may be introduced into a patient. In addition to unconjugated bFGF antibodies and fragments thereof, bFGF antibodies conjugated to toxic agents may also be used. Toxic agent include but are not limited to ricin, doxorubicin, daunorubicin, paclitaxel, ethidium bromide, mitomycin, etoposide, tenoposide, vincristine, vinblastine, colchicine, dihydroxy anthracin dione, actinomycin D, diphteria toxin, Pseudomonas exotoxin (PE)A, PE40, abrin, glucocorticoid and radioisotopes.

The anti-bFGFA antibodies used in the practice of the methods of the invention may be formulated into pharmaceutical compositions comprising a carrier suitable for the desired delivery method. Suitable carriers include any material which when combined with the anti-bFGF mAbs retains the function of the anti-bFGF antibody and is non-reactive with the subject's immune systems. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980).

The anti-bFGF antibody formulations may be administered via any route capable of delivering the antibodies to the target site. Potentially effective routes of administration include, but are not limited to, intravenous, intraperitoneal, intramuscular, intradermal, and the like.

Subjects include humans, monkeys, pigs, apes, dogs, cats, cows, horses, rabbits, mice and rats. Patients included for treatment include but are not limited to individuals with CVD, type 2 diabetes and/or cancer. Human and veterinary treatments may generally involve the repeated administration of the anti-bFGF antibody preparation via an acceptable route of administration such as intravenous injection (IV), at an effective dose. Dosages will depend upon various factors generally appreciated by those of skill in the art. In one embodiment, the daily doses may range from about 1 to 10 mg. Doses in the range of 10-500 mg anti-bFGF antibody per week may be effective and well tolerated, although even higher weekly doses may be appropriate and/or well tolerated. The principal determining factor in defining the appropriate dose is the amount of a particular antibody necessary to be therapeutically effective in a particular context. Repeated administrations may be required in order to achieve inhibition of bFGF. Initial loading doses may be higher. The initial loading dose may be administered as an infusion. Periodic maintenance doses may be administered similarly provided the initial dose is well tolerated.

### ADVANTAGES OF THE INVENTION

Advantages of the invention include the usefulness of the diagnostic or monitoring tests to alert the clinician to pathological complications, which are otherwise difficult to diagnose and treat. For example, the instant invention allows the physician to be alerted of increased risk of coronary heart disease in patients with type 2 diabetes. This can lead to the early application of diagnostic and therapeutic options, which would not otherwise be employed. For example, patients with type 2 diabetes in whom elevated levels of bFGF are detected by the methods of the invention can be treated with anti-bFGF antibodies to decrease the level of bFGF so as to decrease the risk of cardiovascular disease.

### Example 1

Basic fibroblast growth factor (bFGF) is a potent endothelial and smooth muscle cell mitogen which does not normally circulate, Plasma bFGF-like bioactivity was increased in association with persistent micro-albuminuria (a risk marker for cardiovascular disease) in adult type 2 diabetes. Herein, Applicants tested whether baseline plasma bFGF immunoreactivity (IR) predicts the occurrence of a subset of cardiovascular disease outcomes in adults with advanced type 2 diabetes from the Veterans Affairs Diabetes Trial (mean: age 59 yrs, diabetes duration 11 yrs, baseline HbA1c 9.5%). Plasma bFGF-IR was determined with a sensitive and specific two-site enzyme-linked immunoassay in 399 patients at the baseline visit. These results were then evaluated as possible predictors of the occurrence of pre-specified cardiovascular or coronary heart disease endpoints. There was a borderline significant association (p=0.07) between plasma bFGF-IR and the main study cardiovascular disease outcome (myocardial infarction, congestive heart failure, cerebrovascular accident, amputation, cardiovascular death, coronary, cerebrovascular or peripheral revascularization and inoperable coronary artery disease). Plasma bFGF-IR was significantly associated with the occurrence of coronary heart disease (p=0.01). After adjusting for clinical risk factors, bFGF (hazard ratio, HR 1.013; 95% CI 1.007-1.019; p<0.0001), prior macrovascular event (HR 3.55; 95% CI 2.154-5.839; p<0.0001), and duration of diabetes (HR 1.041; 95% CI 1.012-1.071; p=0.0055) were all significantly associated with time to first post-randomization coronary heart disease occurrence. These results suggested that increased plasma bFGF immunoreactivity may be a novel risk marker for cardiovascular disease including coronary heart disease occurrence in adult type 2 diabetes. The type 2 diabetes may be advanced type 2 diabetes.

### Subjects and Methods

### Study Subjects

The study design and clinical inclusion and exclusion criteria for the main VADT have been previously reported (4, 5). Eligible patients without renal insufficiency and without congestive heart failure were randomly assigned to standard vs. intensive glycemic treatment. Randomization was jointly stratified according to baseline insulin use (yes/no) and occurrence of macrovascular event prior to baseline (yes/no). The main VADT study endpoint was a pooled cardiovascular disease outcome that included ischemic coronary artery disease, congestive heart failure, cerebrovascular disease and peripheral arterial disease events. The VADT 465 B substudy pre-specified coronary heart disease (cardiovascular mortality, MI, coronary revascularization, and inoperable CAD) as the primary outcome that would encompass myocardial infarction and closely related ischemic coronary events. Informed consent for the Investigational Review Board-approved substudy was obtained at six outpatient sites from 399 diabetic subjects who had consented to participate in the main VADT. EDTA plasma was then drawn in the morning after an overnight fast at each site. Plasma was aliquoted and shipped frozen (dry ice) to a central laboratory (Maveric, Boston Veterans Affairs Medical Center (VAMC), Boston, Mass.) where it was inventoried and stored at −80° C. Archived, coded frozen EDTA plasma from consecutively enrolled patients was shipped to the laboratory of Dr. Zimering (VA New Jersey Health Care System, Lyons, N.J., (VANJ)) where bFGF-IR assays were performed. All other assays were performed in the Central Laboratory of the VADT (Tufts University, Boston, Mass.). Baseline clinical characteristics are shown in Table 1. All subjects were more than 40 yrs old, and 96% were men,

### Medications

As previously reported, all patients were taking anti-diabetic medications at baseline, including oral agents and/or insulin (4). Patients randomized to the standard or intensive glycemic treatment group were both treated with similar classes of anti-diabetic medications (but at different doses), including the TZD rosiglitazone. Baseline anti-hypertensive medication use included ACE inhibitors in 67% of patients and angiotensin receptor blockers (ARB) in an additional 7% of patients. Statins were used by 62% of patients at baseline. All patients were encouraged to take aspirin 81-162 mg, unless otherwise contraindicated.

### Study Outcomes

Cardiovascular disease outcomes were adjudicated by an independent Study Endpoints Committee as previously described (5). Baseline determination of plasma bFGF-1R (assayed at VANJ) was masked to the information about study endpoint occurrence.

The association of risk factors with time to first post-baseline cardiovascular (CVD) or coronary heart disease (CHD) outcome was modeled using the 399 subjects for whom such post-randomization data was available.

### Laboratory and Clinical Measures

Urinary microalbumin, plasma HbA₁c and urine creatinine were determined by standard methods, and plasma total cholesterol, triglycerides and HDL cholesterol were determined by standardized direct enzymatic assay methods, which methods were previously described (4). Urinary albumin/creatinine ratio was calculated as albumin concentration/creatinine concentration×100. LDL cholesterol was calculated using the Friedenwald equation only for those samples with plasma triglyceride concentration<400 mg/dL. Blood pressure (BP) was recorded in the seated position after a five-minute rest. Three consecutive readings were obtained, and the median value of the three consecutive determinations, computed separately for systolic and diastolic blood pressure, was used for analysis.

### Plasma Samples

### Basic Fibroblast Growth Factor Assays

Basic FGF immunoreactivity (bFGF-IR) in plasma was determined using a sensitive specific two-site enzyme-linked immunoassay (R&D Systems, Inc. Minneapolis, Minn., Product No. HSFB75). Details about the assay performance characteristics have been previously reported (14). Plasma bFGF-IR and bFGF-like bioactivity have previously been shown to be stable for five years or longer at −20° C., and for up to three freeze-thaw cycles (15). Among 43 healthy male blood donors aged 21-63 years old, plasma bFGF-IR was shown to range between 0-4 pg/mL, and there was no effect of age on plasma bFGF level (16).

### Statistics

The VADT substudy estimated that a sample size of 400 subjects would have 90% power for detecting a 50% reduction in the event rates for either the CVD or CHD primary endpoints, which is equivalent to a risk ratio of 2.43 for high vs. low bFGF-IR. The VADT was conducted using the intention to treat principle (5). Both randomization stratification variables—(baseline insulin use and baseline cardiovascular event) and glycemic treatment group were included as covariates in the model when testing for a bFGF effect.

Cox proportional hazards regression analysis was used to model the association between baseline risk factors and time to first post-baseline CHD occurrence. In univariate regression analysis, age, baseline HDL cholesterol concentration, baseline nephropathy, baseline creatinine concentration, and duration of diabetes were each significantly associated (p<0.0001) with time to first CHD occurrence. These and other risk variables (non-Hispanic white race, baseline fibrinogen concentration) significantly associated (p<0.05) with time to first CHD event were included as covariates in models that tested for a bFGF effect. Backward elimination was used to obtain the best fit model using an alpha level of ≦0.05 as the cutoff for variable retention in the final model. Baseline ACE inhibitor use, and angiotensin receptor blocker (ARB) each had a borderline significant association (p≦0.2) with time to first CHD event and were included as covariates in models that tested for a bFGF effect. Baseline glycosylated hemoglobin (p=0.98), current cigarette smoking (p=0.87) and baseline plasminogen activator inhibitor-1 (PAI-1) concentration (p=0.57) were not significantly associated with time to first post-baseline CHD occurrence in univariate regression analysis and so were not included as covariates in models that tested for a bFGF effect.

### Results

### Baseline and Follow-Up Characteristics in the Study Patients

Our subject group included 57% non-Hispanic white, 21% African-American, and 18% Hispanic patients. Thirty-seven percent of the study subjects reported a prior macrovascular event at study entry (Table 1). Study treatment was associated with significant decreases in glycosylated hemoglobin, systolic blood pressure, plasma total cholesterol, LDL cholesterol, triglycerides and current cigarette smoking after five years compared to baseline levels (Table 1; p<0.0001). Body weight, BMI, waist-hip ratio, plasma HDL cholesterol, and serum creatinine concentration all increased significantly after five years compared to baseline levels (Table 1).

### Frequency of Occurrence of Pooled Endpoints

Eighty-four first CHD events occurred in 84 patients during an average of 6 years of VADT study treatment (Table 2). One-hundred ten “first” CVD events occurred in 110 patients over the same time period including 16 cases of congestive heart failure, 12 cases of cerebrovascular disease, and 4 cases of peripheral arterial disease (Table 2). There was a borderline significant association (p=0.07) between baseline plasma bFGF and time to first post-baseline CVD occurrence and the strength of this association was not substantially affected by adjustment for prior cardiovascular event or glycemic treatment.

Association Between Plasma bFGF and First Post-Baseline Occurrence of CHD

Plasma bFGF was significantly associated with time to first post-baseline CHD occurrence (p=0.01). The best fit model of risk factors associated with the time to first CHD occurrence during up to 7.5 years of follow-up had as significant predictors: baseline plasma bFGF (hazard ratio, HR 1.013; p<0.0001), prior cardiovascular event (HR 3.547; p<0.0001), and duration of diabetes (HR 1.041; p=0.0055) (Table 3). There was no significant interaction between bFGF and either diabetes duration, or glycemic treatment.

Comparison of Plasma bFGF Level Effect on CHD Event Occurrence

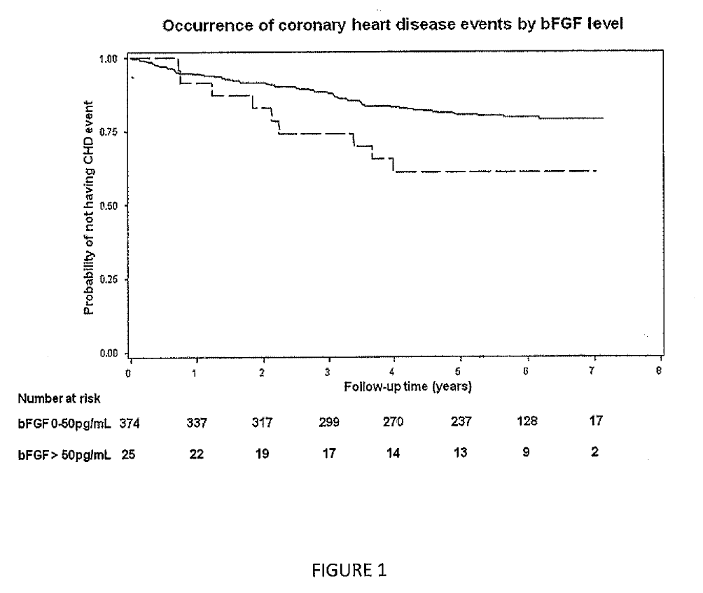

A basic fibroblast growth factor concentration above 50 pg/mL induces more than half-maximal stimulation of proliferation in endothelial cells, and is required to cause significant migration and proliferation in smooth muscle cells in vitro (6). We compared the survival curves for time to occurrence of a coronary heart disease event in groups of patients with baseline bFGF 0-50 pg/mL vs bFGF>50 pg/mL (FIG. 1). There was a statistically significant between-group difference (p=0.03) in the time to occurrence of coronary heart disease (FIG. 1). At a bFGF level>50 pg/mL roughly two-thirds of the total events had occurred after 2.5 years of study treatment and no further events occurred after 4 years follow up (FIG. 1). At bFGF levels less than 50 pg/mL events continued to occur up to 6 years after initiation of study treatment. The survival curves for the comparison of the effect of the two bFGF levels separate after approximately two years and reach their maximal separation after about 4 years of study treatment (FIG. 1).

Effect of One Year Study Treatment on Plasma bFGF

We were able to obtain measurement of plasma bFGF one year after initiation of study treatment in a subgroup of 215 consecutively enrolled subjects including nearly equal numbers of patients randomized to either standard or intensive glycemic treatment. Baseline bFGF did not differ significantly for patients in the Standard vs. Intensive groups (Table 4). Mean plasma bFGF decreased significantly after one year in both INT and STD treated patients (−32% and −46%), and the one-year bFGF change was not statistically significantly different for INT vs. STD treatment (Table 4).

Association Between Study Treatment Effect on bFGF and CHD Occurrence

The one-year bFGF average was not significantly associated with the time to occurrence of CHD (p=0.157). There was no significant association between the one year difference in bFGF and CHD occurrence even after adjusting for prior cardiovascular event or glycemic treatment.

### Discussion

The present data are the first to suggest an association between increased baseline plasma bFGF and the occurrence of coronary heart disease in adults with long-standing type 2 diabetes. The increased risk for CVD or CHD in patients with high baseline bFGF persisted during 5-7.5 years of study treatment despite substantial improvements in the levels of most traditional cardiovascular risk factors, and this significant association with CVD or CHD remained after adjusting for standard vs. intensive glycemic treatment, ACE inhibitor or ARB medication use, presence of a baseline cardiovascular event, and diabetes duration. The data are consistent with the possibility that substantially increased plasma bFGF may have a long-lasting effect on important mechanisms underlying cardiovascular disease risk in older adults with advanced type 2 diabetes.

Basic FGF is one of the most potent known angiogenic factors (17). However, lacking an amino terminal signal sequence necessary for efficient release from cells (9), bFGF does not normally circulate. Basic FGF is present in endothelial cells, as well as in the extracellular matrix of vascular tissues where it is bound to heparan sulfate proteoglycan. Following arterial vascular injury, bFGF is released locally and is thought to stimulate smooth muscle cell migration and proliferation important for neointimal formation (8).

Plasma bFGF increases in a number of different cancers including renal cancer (18), consistent with kidney as a source of bFGF which can promote local tumor cell proliferation and angiogenesis (6). Substantially increased plasma bFGF was also reported in children suffering from HIV with nephropathy or hemolytic uremic syndrome (19). In the latter syndrome very high bFGF levels were postulated to reflect widespread endothelial injury and thrombosis (19). Mean concentrations of plasma bFGF in the current study group are near the level reported to cause half-maximal proliferation in endothelial cells in vitro (6). Substantially higher concentrations of plasma bFGF (>50 pg/mL) are consistent with doses reported to cause significant proliferation in smooth muscle cells (6). Taken together these data suggest that markedly increased bFGF, a locally-acting mitogen normally involved in wound healing, may be capable of acting via the systemic circulation to promote cell proliferation and cell migration in atheromatous tissue which expresses FGF receptor (20).

The tissue sources of increased plasma bFGF in obese type 2 diabetes are not known. We reported a significant association between increased plasma bFGF and waist-hip ratio in the present VADT subset (14). Human omentum is highly vascular, it contains substantial concentrations (2 ug/g) of a highly bioactive bFGF-like protein (21). In addition, omental preadipocytes express high levels of bFGF messenger RNA (22). Whether bFGF is released into the general circulation from omentum, and the mechanisms, governing its release in viscerally-obese type 2 diabetes, remain unanswered. Macrophages abundant in visceral fat secrete proteases capable of liberating bFGF from the extracellular matrix (23). Pro-inflammatory visceral adipocytokines (tumor necrosis factor-alpha, interleukin-1 and interferon-gamma) can substantially increase bFGF release from microvascular endothelial cells in vitro (24). The release of pro-inflammatory adipocytokines is known to be regulated by angiotensin II (25) which itself increases bFGF expression in vascular smooth muscle cells (26). Thus angiotensin II-adipocytokine interactions may play a role in the amplification of plasma bFGF release from one or more injured microvascular beds in obese type 2 diabetic patients.

Poor glycemic control has been associated with several markers of inflammation, endothelial dysfunction and/or oxidative stress, including: PAI-1, C-reactive protein, TNF-alpha, IL-6, adhesion molecules and reactive oxygen species (27). Plasma and urinary levels of another broad-spectrum growth regulator, i.e. transforming growth factor beta, have been shown to be associated with poor glucose control, elevated PAI-1, increased BMI and diabetic nephropathy (28). Existing data, although scant, suggests at least four reasons why it is unlikely that the present association between increased plasma bFGF and CHD occurrence is due to confounding by an association between bFGF and a marker of systemic inflammation. First, log baseline bFGF was significantly inversely associated with baseline glycosylated hemoglobin in the current study group (14). Second, the pro-coagulant, pro-inflammatory protein fibrinogen was modestly associated with CHD risk in univariate regression analysis, but had no significant effect on the association between bFGF and CHD. Third, Larsson et al. reported no significant association (in human donor serum) between serum bFGF and either high sensitivity C-reactive protein or serum amyloid protein, two inflammatory markers of cardiovascular risk (29). Finally, C-reactive protein, IL-6, and several other inflammatory markers of CHD risk are positively associated with BMI (27), whereas there was no significant association between bFGF and BMI (14).

The results were consistent with a prior report that plasma bFGF was increased in patients with early coronary artery disease (11). Prior evidence from a number of sources suggests that FGF expression may play a unifying role in mediating diverse hemodynamic, dyslipidemic, angiogenic, or prothrombotic effects on atherosclerotic vascular intimal proliferation. First, FGF expression in smooth muscle cells was increased by cholesterol esters (30), and decreased by HDL (31). Second, hemodynamic stress induces vascular smooth muscle cell expression of bFGF (32). Third, basic FGF may act as a local angiogenesis factor to promote plaque neovascularization (33). Fourth, basic FGF induces the endothelial cell synthesis of PAI-1 (34)—a major inhibitor of fibrinolysis and a risk marker for myocardial infarction (35). Fifth, basic FOP inhibits smooth muscle cell type 1 collagen synthesis and stimulates collagenase production-effects which may contribute to plaque instability (36). Basic FGF expression in certain vascular tissues activates coordinated gene expression which normally functions to permit local tissue remodeling (36, 37). The present data taken together with the known diverse effects of bFGF in the vessel wall lead us to suggest the novel hypothesis that substantial concentrations of systemically bioavailable bFGF can promote atherosclerosis, thrombosis, plaque neovascularization and/or plaque instability.

The lack of association between glycemic treatment and CHD risk in the substudy group is consistent with findings from the main VADT that intensive glycemic treatment per se was not associated with a lower overall risk for CVD (5). Despite a prior significant association between increased plasma bFGF and persistent micro-albuminuria (10), neither nephropathy nor elevated serum creatinine was significantly associated with CHD occurrence after adjusting for duration of diabetes. These data are consistent with the possibility that a threshold level of endothelial cell injury underlying micro-albuminuria may be required to increase plasma bFGF (10), but CHD risk associated with substantially increased bFGF differs from the risk associated with nephropathy.

VADT treatment resulted in substantial improvements in blood pressure, lipid levels and glycemia in the majority of patients randomized to either glycemic treatment arm. This may have contributed to the lack of an association between one-year bFGF change and glycemic treatment assignment. Significant weight gain resulted from improved glycemic control in the VADT (5). Weight gain, fluid retention, or a specific treatment effect may have contributed to cases of congestive heart failure possibly weakening an underlying significant association between plasma bFGF and CVD. The model suggests that a 50 pg/mL increase in baseline plasma bFGF is associated with a 1.9-fold increase in the hazard ratio for CHD. Whether the one-year average bFGF can predict the longer-term (10-12 years) post-baseline occurrence of CHD will be studied in the ongoing Veterans Affairs Diabetes Trial Follow-up Study.

The incidence of obesity and type 2 diabetes in the United States and worldwide is increasing at an alarming rate (38) and ethnic minorities in the U.S suffer from the highest rates of diabetes (39). A strength of the study is that it included a substantial proportion of African-American and Hispanic adults who have an increased prevalence of type 2 diabetes. A limitation of the study is that the findings may only reflect the experience of middle-aged and older obese men with long-standing, poorly controlled, type 2 diabetes. More study in women and young adults with type 2 diabetes is needed to determine whether plasma bFGF may indicate an increased risk for first CHD events among patients who may be at substantially lower risk for CHD than was seen in those subjects in the high risk study group.

In summary, the current findings suggest that increased plasma basic fibroblast growth factor was associated with a substantially increased 5-year risk for CVD or CHD occurrence in older men with advanced type 2 diabetes. Plasma bFGF is a potentially useful marker of increased risk for CVD or CHD in adult type 2 diabetes. Although the association demonstrated here does not prove causality, it suggests a novel mechanism in which bFGF may act via the general circulation to contribute to a substantially increased risk for cardiovascular coronary heart disease, the leading cause of death in adults with type 2 diabetes.

TABLE 1
Baseline and follow up characteristics in the 399 study patients

| Variable | Baseline | Follow up* | p-value |
|---|---|---|---|
| Age | 59.3 ± 8.5 | | |
| Diabetes duration | 11.4 ± 7.6 | | |
| Male | 96% | | |
| Prior CV event | 37% | | |
| Hypertension | 70% | | |
| Race | | | |
| Non-Hispanic White | 57% | | |
| African-American | 21% | | |
| Hispanic | 18% | | |
| Current smoking | 18% | 12% | <0.0001 (n = 397) |
| bFGF (pg/mL) | 15.1 ± 25.8 (n = 399) | 8.0 ± 16.3\hat{ }(n = 215) | 0.0014 (n = 215) |
| HbA₁C (%) | 9.5 ± 1.4 | 8.2 ± 1.6 | <0.0001 (n = 396) |
| Systolic bp (mm Hg) | 130.9 ± 17.4 | 126.9 ± 17.3 | 0.0002 (n = 393) |
| BMI (kg/m²) | 30.9 ± 4.6 | 32.3 ± 5.7 | <0.0001 (n = 397) |
| Weight (lbs) | 212.7 ± 37.3 | 221.5 ± 43.5 | <0.0001 (n = 397) |
| Waist/hip ratio | 0.991 ± 0.07 | 0.998 ± 0.07 | 0.0171 (n = 364) |
| Total cholesterol (mg/dL) | 183 ± 44 | 156 ± 41 | <0.0001 (n = 379) |
| LDL cholesterol (mg/dL) | 108 ± 33 | 83 ± 33 | <0,0001 (n = 369) |
| HDL cholesterol (mg/dL) | 37 ± 10 | 41 ± 12 | <0.0001 (n = 379) |
| Triglycerides (mg/dL) | 195 ± 175 | 158 ± 122 | <0.0001 (n = 379) |
| Serum creatinine (mg/dL) | 0.99 ± 0.2 | 1.2 ± 0.5 | <0.0001 (n = 393) |

Results for continuous variables are displayed as mean +/− SD;
*year 5 or last annual visit prior to study termination;
\hat{ }year 1 annual visit

TABLE 2
First post-randomization cardiovascular disease (CVD)
events in 399 study patients.

| Individual events | n = 110 |
|---|---|
| Congestive heart failure | 16 |
| Amputation | 3 |
| Peripheral revascularization | 1 |
| Cerebrovascular accident | 7 |
| Cerebrovascular revascularization | 5 |
| Myocardial infarction | 28 |
| Coronary revascularization | 44 |
| Inoperable coronary artery disease | 3 |
| Cardiovascular death | 3 |

Coronary heart disease (myocardial infarction n=28, coronary revascularization n=49, inoperable coronary artery disease n=4 or cardiovascular death n=3) was the first post-randomization event in 84 subjects.

TABLE 3
Cox proportional hazard regression: time to first post-baseline CHD event

| Variable | HR | 95% CI | p-value |
|---|---|---|---|
| Baseline bFGF | 1.013 | 1.007-1.019 | <0.0001 |
| Prior CV even | 3.547 | 2.154-5,839 | <0.0001 |
| Duration of | 1.041 | 1.012-1.071 | 0.0055 |
| diabetes | | | |

n = 375 subjects;
HR—hazard ratio,
CI—confidence interval

TABLE 4
One -year change in plasma bFGF by treatment in 215 study patients
Mean plasma bFGF

| Treatment | Baseline | Year 1 | % change | (95% CI) | p-value |
|---|---|---|---|---|---|
| Standard (108) | 15.0 | 8.1 | −46 | (−8, −84) | 0.35 |
| Intensive (107) | 11.8 | 8.0 | −32 | (−5, −60) | |

Number of subjects indicated in parentheses.
CI—confidence intervals
p = 0.35 for comparison of mean change in bFGF, comparing Standard vs Intensive

### REFERENCES

1. Morbidity Mortality Weekly Report. 2001. Mortality from coronary heart disease and acute myocardial infarction—United States 1998; 50(06): 90-93.
2. Stamler J, Vaccaro O, Neaton J, Wentworth D, for the Multiple Risk Factor Intervention Trial Research Group. Diabetes, other risk factors, and 12-year cardiovascular mortality for men screened in the Multiple Risk Factor Intervention Trial, Diabetes Care 1993; 16: 434-44.
3. Folsom A R, Chambless L E, Duncan B B, Gilbert A C, Pankow J S, and the Atherosclerosis Risk in Communities Study Investigators. Prediction of coronary heart disease in middle-aged adults with diabetes. Diabetes Care 2003; 26 (10): 2777-2784.
4. Abraira C, Duckworth W, McCarren M, Emanuele N, Arca D, Reda D, Henderson W. Design of the cooperative study of glycemic control and complications in diabetes mellitus type 2. J Diab & Cowl 2003; 17: 314-322.
5. Duckworth W, Abraira C, Moritz T, Reda D, Emanuele N, Reaven P D, Zieve F J, Marks J, Davis S N, Hayward R, Warren S R, Goldman S, McCarren M, Vitek M E, Henderson W G, Huang G D; VADT Investigators. Glucose control and vascular complications in veterans with type 2 diabetes. N Engl J Med. 2009; 360(2): 129-139. Erratum in: N Engl J Med., 2009 Sep. 3; 361(10):1024-5; N Engl J Med., 2009 Sep. 3; 361(10):1028
6. Gospodarowicz D, Ferrara N, Schweigerer L, Neufeld G. Structural characterization and biological functions of fibroblast growth factor. Endocr Rev. 1987: 8: 95-114.
7. Sato Y, Hamanaka R, Ono J, Kuwano M, Rifkin D B, Takaki R. The stimulatory effect of PDGF on vascular smooth muscle cell migration is mediated by the induction of endogenous basic FGF, Biochem Biophys Res Commun. 1991; 174(3): 1260-1266.
8. Lindner V, Reidy M A. Proliferation of smooth muscle cells after vascular injury is inhibited by an antibody against basic fibroblast growth factor. Proc. Natl. Acad. Sci. USA 1991; 88: 3739-3743.
9. Esch F, Baird A, Ling N, Ueno N, Hill F, Denoroy L, Klepper R, Gospodarowicz D, Bohlen P, Guillemin R. Primary structure of bovine pituitary basic fibroblast growth factor (FGF) and comparison with the amino-terminal sequence of bovine brain acidic FGF. Proc Nat Acad Sci (USA) 1985; 19: 6507-6511.
10. Zimering M. B, Eng J. Increased basic fibroblast growth factor-like substance in plasma from a subset of middle-aged or elderly male diabetic patients with microalbuminuria or proteinuria. J Clin Endo Metab. 1996; 81: 4446-4452.
11. Hasdai D, Barak V, Leibovitz E, Herz I, Sclarovsky 5, Eldar M, Scheinowitz M. Serum basic fibroblast growth factor levels in patients with ischemic heart disease. Int J Cardiol. 1997; 59(2): 133-138.
12. Deckert T, Feldt-Rasmussen B, Borch-Johnsen K, Jensen T, Kofoed-Enevoldsen A. Albuminuria reflects widespread vascular damage: the Steno hypothesis. Diabetologia 1989; 32: 219-226.
13. Valmadrid C T, Klein R, Moss S E, Klein B E. The risk of cardiovascular disease mortality associated with microalbuminuria and gross proteinuria in persons with older-onset diabetes mellitus. Arch Intern Med. 2000; 160(8): 1093-1100.
14. Zimering M B, Anderson R J, Luo P, Moritz T E; Investigators for the Veterans Affairs Diabetes Trial. Plasma basic fibroblast growth factor is correlated with plasminogen activator inhibitor-1 concentration in adults from the Veterans Affairs Diabetes Trial. Metabolism 2008; 57(11): 1563-1569.
15. Zimering M B, 2002. Effect of intravenous bisphosphonates on release of basic fibroblast growth factor in serum of patients with cancer-associated hypercalcemia. Life Sciences 2002; 70: 1947-1960,
16. Larsson A, Skoldenberg E, Ericson H. Serum and plasma levels of FGF-2 and VEGF in healthy blood donors. Angiogenesis 2002; 5: 107-110.
17. Schweigerer L, Neufeld G, Friedman J, Abraham J A, Fiddes J C, Gospodarowicz D. Capillary endothelial cells express basic fibroblast growth factor, a mitogen that promotes their own growth. Nature 1987; 325: 257-259,
18, Dirix L Y, Vermeulen P B, Pawinski A, Prové A, Benoy I, De Pooter C, Martin M, Van Oosterom A T. Elevated levels of the angiogenic cytokines basic fibroblast growth factor and vascular endothelial growth factor in sera of cancer patients. Br J Cancer. 1997; 76(2): 238-243.
19. Ray P E, Liu X H, Xu L, Rakusan T. Basic fibroblast growth factor in HIV-associated hemolytic uremic syndrome. Pediatr Nephrol, 1999; 13(7): 586-593.
20. Hughes S E, Crossman D, Hall P A. Expression of basic and acidic fibroblast growth factors and their receptor in normal and atherosclerotic human arteries. Cardiovasc Res. 1993; 27(7): 1214-1219.
21. Mydlo J H, Kral J G, Macchia R J. Differences in prostate and adipose tissue basic fibroblast growth factor: analysis of preliminary results. Urology 1997; 50(3): 472-478.
22. Teichert-Kuliszewsak K, Hamilton B S, Deitel M, Roncari D A. Augmented production of heparin-binding mitogenic proteins by preadipocytes from massively obese persons. J Clin Invest 1992; 90: 1226-1231.
23. Liuzzo J P, Petaneeska S S, Moscatelli D, Devi L A, 1999. Inflammatory mediators regulate cathepsin S in macrophages and microglia: A role in attenuating heparan sulfate interactions. Mol Med. 1999; 5(5): 320-333.
24, Samaniego F, Markham P D, Gendelman R, Gallo R C, Ensoli B. Inflammatory cytokines induce endothelial cells to produce and release basic fibroblast growth factor and to promote Kaposi's sarcoma-like lesions in nude mice. J Immunol. 1997; 158(4): 1887-1894.
25. Kurata A, Nishizawa H, Kihara S, Maeda N, Sonoda M, Okada T, Ohashi K, Hibuse T, Fujita K, Yasui A, Hiuge A, Kumada M, Kuriyama H, Shimomura I, Funahashi T. Blockade of angiotensin II type-1 receptor reduces oxidative stress in adipose tissue and ameliorates adipocytokine dysregulation. Kidney Int. 2006; 70(10): 1717-1724.
26. Itoh H, Mukoyama, M, Pratt R, Gibbons G, Dzau V. Multiple autocrine growth factors modulate vascular smooth muscle cell growth response to angiotensin II. J. Clin Invest 1993; 91: 2268-2274.
27. Goldberg, R B. Cytokine and cytokine-like inflammation markers, endothelial dysfunction and imbalanced coagulation in development of diabetes and its complications J Clin Endo Metab 2009; 94: 3171-3182.
28. Sharma K, Ziyadeh F N. Hyperglycemia and diabetic kidney disease. The case for transforming growth factor-beta as a key mediator. Diabetes 1995; 44(10): 1139-1146.
29. Larsson A, Hansson L O. High sensitivity CRP and serum amyloid A as expressions of low grade inflammation do not correlate with bFGF or VEGF. Ups J Med Sci. 2003; 108(1): 51-59.
30, Kraemer R, Pomerantz K B, Joseph-Silverstein J, Hajjar D P. Induction of basic fibroblast growth factor mRNA and protein synthesis in smooth muscle cells by cholesterol ester enrichment and 25-hydroxycholesterol. J Biol Chem. 1993; 268(11): 8040-8045,
31. Cucina A, Scavo M P, Muzzioli L, Coluccia P, Ceccarini S, Fuso A, Cavallaro A. High density lipoproteins downregulate basic fibroblast growth factor production and release in minimally oxidated-LDL treated smooth muscle cells. Atherosclerosis. 2006; 189(2): 303-309.
32. Gosgnach, W, Challah, M, Coulet, F, Michel, J-B, Battle, T. Shear stress induces angiotensin converting enzyme expression in cultured smooth muscle cells: possible involvement of bFGF. Cardiovasc Research 2000; 45(2): 486-492.
33. Hughes S E. Localisation and differential expression of the fibroblast growth factor receptor (FGFR) multigene family in normal and atherosclerotic human arteries. Cardiovasc Res. 1996; 32(3): 557-569.
34. Kaneko T, Fujii S, Matsumoto A, Goto D, Ishimori N, Watano K, Furumoto T, Sugawara T, Sobel B E, Kitabatake A. Induction of plasminogen activator inhibitor-1 in endothelial cells by basic fibroblast growth factor and its modulation by fibric acid. Arterioscler Thromb Vasc Biol. 2002; 22(5): 855-860.
35. Thogersen A M, Jansson J H, Boman K, Nilsson T K, Weinehall L, Huhtasaari F, Hallmans G. High plasminogen activator inhibitor and tissue plasminogen activator levels in plasma precede a first acute myocardial infarction in both men and women: evidence for the fibrinolytic system as an independent primary risk factor. Circulation 1998; 98(21): 2241-2247.
36. Pickering J G, Ford C M, Tang B, Chow L H. Coordinated effects of fibroblast growth factor-2 on expression of fibrillar collagens, matrix metalloproteinases, and tissue inhibitors of matrix metalloproteinases by human vascular smooth muscle cells. Evidence for repressed collagen production and activated degradative capacity. Arterioscler Thromb Vasc Biol. 1997; 17(3): 475-482.
37. Okamura K, Sato Y, Matsuda T, Hamanaka R, Ono M, Kohno K, Kuwano M. Endogenous basic fibroblast growth factor-dependent induction of collagenase and interleukin-6 in tumor necrosis factor-treated human microvascular endothelial cells. J Biol Chem. 1991; 266(29): 19162-19165.
38. Wild 5, Roglic G, Green A, Sicree R, King H. Global prevalence of diabetes: estimates for the year 2000 and projections for 2030. Diabetes Care 2004; 27(5): 1047-1053.
39. Cowie C C, Rust K F, Byrd-Holt D D, Eberhardt M S, Flegal K M, Engelgau M M, Saydah S H, Williams D E, Geiss L S, Gregg E W. Prevalence of diabetes and impaired fasting glucose in adults in the U.S. population: National Health and Nutrition Examination Survey 1999-2002. Diabetes Care. 2006; 29(6): 1263-1268.

### Example 2

Baseline plasma bFGF immunoreactivity (IR) is measured directly or indirectly. Direct measurement is carried out by the addition of specific analyte that comprises antibody or a combination of several antibody to the sample being tested. The complex so formed between the plasma bFGF and the analyte is directly quantitatively measured. In the indirect assay the complex so formed is removed and the remaining analyte is measured. In indirect method, the quantitative measurement is inversely proportional to the amount of bFGF in the sample.

### Subjects and Methods

### Study Subjects

The study design and clinical inclusion and exclusion criteria for the main VADT have been previously reported (13). Patients with renal insufficiency or congestive heart failure were excluded from participation. Randomization was jointly stratified according to baseline insulin use (yes/no), and occurrence of macrovascular event prior to baseline (yes/no), and VA site. The main VADT study endpoint was a pooled cardiovascular disease outcome encompassing ischemic coronary artery disease, congestive heart failure, cerebrovascular disease, and peripheral arterial disease events. Informed consent for the Investigational Review Board-approved substudy was obtained at six outpatient sites from 399 diabetic subjects who had consented to participate in the main VADT. EDTA plasma was then drawn in the morning after an overnight fast. Plasma was aliquoted and shipped frozen (dry ice) to a central laboratory (Maveric, Boston Veterans Affairs Medical Center (VAMC), Boston, Mass.) where it was inventoried and stored at −80° C. Archived, coded frozen EDTA plasma from consecutively enrolled patients was shipped to the laboratory of Dr. Zimering (VA New Jersey Health Care System, Lyons, N.J., (VANJ)) where bFGF-IR assays were performed. All other assays were performed in the Central Laboratory of the VADT (Tufts University, Boston, Mass.). All subjects were more than 40 yrs old, and 96% were men.

### Medications

As previously reported, all patients were taking anti-diabetic medications at baseline, including oral agents and/or insulin (13). Patients randomized to the standard or intensive glycemic treatment group were both treated with similar classes of anti-diabetic medications (but at different doses), including the TZD rosiglitazone. Baseline anti-hypertensive medication use included ACE inhibitors in 67% of patients and angiotensin receptor blockers (ARB) in an additional 7% of patients. Statins were used by 62% of patients at baseline.

### Study Outcomes

Cardiovascular disease outcomes were adjudicated by an independent Study Endpoints Committee as previously described (5). Cardiovascular disease (CVD) is defined as myocardial infarction, congestive heart failure, cerebrovascular accident, amputation, cardiovascular death, coronary revascularization, cerebrovascular revascularization, peripheral revascularization or inoperable coronary artery disease. Coronary heart disease (CHD) is defined as myocardial infarction, coronary revascularization, inoperable coronary artery disease or cardiovascular death. Baseline (n=399) or year 1 determination of plasma bFGF-IR in a randomly selected subset (n=215, chosen due to budgetary constraints) of baseline patients was masked to the information about study endpoint occurrence. The association of risk factors with time to first post-baseline cardiovascular (CVD) or coronary heart disease (CHD) outcome was modeled using the 399 subjects for whom such post-randomization data was available.

### Laboratory and Clinical Measures

Urinary microalbumin or creatinine and plasma HbA₁c, total cholesterol, triglycerides and HDL cholesterol were determined by standardized direct enzymatic assay methods (13). Blood pressure (BP) was recorded in the seated position after a five-minute rest. The median value of three consecutive determinations, computed separately for systolic and diastolic blood pressure, was used for analysis.

### Plasma Samples

### Basic Fibroblast Growth Factor Assays

Basic FGF immunoreactivity (bFGF-IR) in plasma was determined using a sensitive specific two-site enzyme-linked immunoassay (R&D Systems, Inc. Minneapolis, Minn.) as previously reported (14). Plasma bFGF-IR and bFGF-like bioactivity have previously been shown to be stable for five years or longer at −20° C., and for up to three freeze-thaw cycles (14). Plasma bFGF-IR ranged between 0-4 pg/mL in healthy male volunteer blood donors, and there was no effect of age on plasma bFGF level (15).

### Statistics

The VADT was conducted using the intention to treat principle (5). The VADT substudy estimated that a baseline sample size of 400 subjects would have 90% power for detecting a 50% reduction in the event rates for either the CVD or CHD primary endpoints, which is equivalent to a risk ratio of 2.43 for high vs. low bFGF-IR. Baseline insulin use and baseline cardiovascular event—the randomization stratification variables—and glycemic treatment group were included as covariates in models when testing for a bFGF effect. Cox proportional hazards regression analysis was used to model the association between baseline risk factors and time to first post-baseline CHD, or CVD occurrence (7). Risk variables previously shown to be significantly associated (p<0.05) with time to first CVD or CHD event occurrence in univariate analysis (7), were included as covariates in models that tested for a bFGF effect. Backward elimination was used to obtain the best fit model using an alpha level of <0.05 as the cutoff for variable retention in the final model.

Based on our prior work, we hypothesized that increased bFGF determined at year 1 may reflect suboptimal blockade of the renin angiotensin system (8) which is a known risk factor for CVD events (16). Consistent with our prior methodology (17), year 1 bFGF was dichotomized at the upper limit of normal reported in adult men (4.4 pg/mL) (15) in models of the risk factors associated with time to first post-year 1 CVD occurrence.

### Results

### Baseline Characteristics and Outcomes in the Study Patients

Our subject group was comprised of men having the following means: age 59 years; diabetes duration 11.4 years; hemoglobin A₁c 9.5%; BMI 31 kg/m²; and 37% of whom reported a prior macrovascular event at study entry (Table 5). One hundred-five first CVD events occurred in 105 patients during an average of 6 years of VADT study treatment.

### Changes in Risk Factor Levels During Study Treatment

Study treatment was associated with significant decreases in glycosylated hemoglobin, and in several known risk factors for CVD including systolic blood pressure and plasma LDL cholesterol concentration (Table 5; p<0.0001). Body weight, BMI, plasma HDL cholesterol, and serum creatinine concentration all increased significantly after three years compared to baseline levels (Table 5).

Association Between Plasma bFGF and First Post-Baseline Occurrence of CHD or CVD

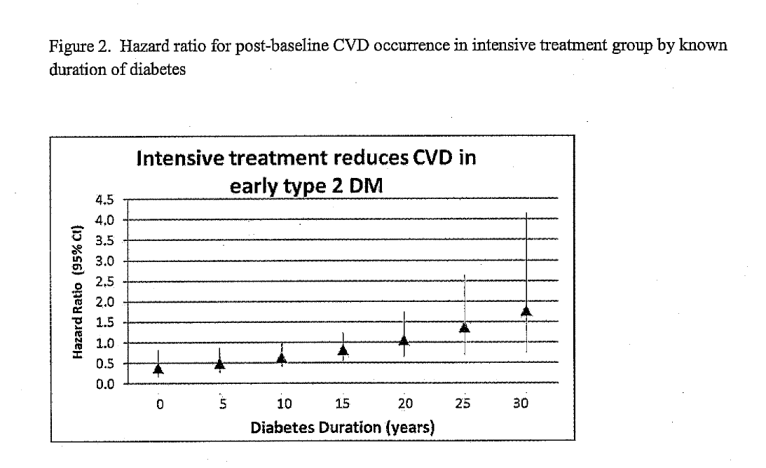

The best fit model of risk factors associated with the time to first CHD occurrence during up to 7.5 years of follow-up had as significant predictors: baseline plasma bFGF (hazard ratio, HR 1.013; p<0.0001), prior cardiovascular event (HR 3.547; p<0.0001), and duration of diabetes (HR 1.041; p=0.0055), as previously reported (7). There was no significant interaction between bFGF and either diabetes duration, or glycemic treatment (7). In the current model that included adjustment for a significant duration-treatment interaction (p=0.019), bFGF (hazard ratio, HR 1.012; p=0.0003), and prior cardiovascular event (HR 3.488; p<0.0001) were still significant predictors of time to first CHD occurrence (Table 6). The best fit model of risk factors associated with the time to first post-randomization occurrence of the main study CVD endpoint (n=105 events in 399 subjects) had as significant predictors: baseline bFGF (p=0.01), duration-treatment interaction (p=0.03), age (p=0.03), and prior CV event (p<0.0001) (Table 7). The adjusted model that includes a significant ‘treatment×duration’ interaction term predicted significantly decreased CVD hazard ratios (0.38-0.63) associated with intensive glucose-lowering in patients with ten or fewer years of baseline known diabetes duration (FIG. 2) and CVD hazard ratios not significantly different from 1 (0.82-1.78) for durations of fifteen years or longer (FIG. 2).

Association Between Increased Year 1 bFGF and CVD

As previously reported, mean year 1 plasma bFGF decreased significantly in STD- or INT-treated patients (8.1 or 8.0 pg/mL) compared to baseline bFGF levels (15.0 or 11.8 pg/mL) (7). There was no significant association between increased year 1 bFGF and time to first post-year 1 CHD (p=0.21) or CVD (p=0.19) occurrence among the 215 subjects tested for bFGF at year 1. In a high risk subset with lengthy (a. 15 years) baseline duration of diabetes, there was a nearly significant association between increased year 1 bFGF and time to post-year 1 CVD occurrence (n=22 events in 71 patients, HR 2.439, p=0.05).

### Discussion

The current findings are consistent with earlier studies suggesting a possible interaction between intensive treatment and diabetes duration in the risk for cardiovascular disease in adult type 2 diabetes. For example, in newly-diagnosed diabetic patients from the UKPDS there was a trend toward a significant overall cardiovascular benefit from intensive treatment (18). The absence of a similar beneficial effect from intensive glucose-lowering in the ADVANCE, ACCORD or VADT may have been due (in part) to more advanced type 2 diabetes enrolled in the latter three studies. The ADVANCE trial had a lower mean separation in glycosylated hemoglobin (INT vs STD groups) compared to ACCORD or VADT (19). It is not clear whether less intensive glucose-lowering in ADVANCE subjects despite shorter mean diabetes duration (8.0 years) compared to subjects in ACCORD (10 years) or VADT (11.5 years) contributed to a non-significant trend toward lower total mortality for intensive glucose-lowering in ADVANCE (incidence rate ratio: IRR 0.93; p=0.27) (19).

The underlying mechanism(s) for a significant treatment-duration interaction in the risk for cardiovascular disease in type 2 diabetes is unclear. One possibility suggested by the VADT substudy of Reaven et al. (6) is that factors associated with atherosclerosis reflected by the coronary artery calcium score may contribute to cardiovascular disease occurrence associated with intensive glucose-lowering. Our results (from a different VADT patient subgroup) are in agreement with those of Reaven et al. (6), that reduced cardiovascular disease occurrence associated with intensive glucose-lowering in patients with ten or fewer years of diabetes duration may be due in part to less extensive baseline atherosclerosis. Still other unidentified factors may act in concert with underlying atherosclerosis to contribute to the increased CVD risk in longstanding type 2 diabetes.

One such factor may be basic FGF, a potent angiogenic and smooth muscle cell mitogen (20) which is released following endothelial injury (21), and which is capable of inducing smooth muscle cell migration and proliferation important in neointima formation (22, 23). Plasma basic FGF is low or undetectable in healthy subjects (24), but increases in the presence of coronary artery disease (25). Plasma bFGF-like bioactivity decreases after treatment with angiotensin converting-enzyme inhibitor medications in micro-albuminuric adult type 2 DM (8), perhaps consistent with a role for increased plasma bFGF in the global CVD risk associated with renin-angiotensin system (RAS) activation (17). Since bFGF can act as a local mediator of the atherosclerotic vascular effects of dyslipidemia (26, 27), and hemodynamic stress (28) or can stimulate the endothelial cell release of PAI-1 (29) which is a major inhibitor of fibrinolysis, bFGF may contribute to CVD encompassing both atherothrombosis and events associated with vascular hypertrophy (30). The present findings are consistent with this possibility, since increased post-baseline bFGF nearly significantly predicted CVD occurrence in a high risk subset with 15 years or longer baseline diabetes duration.

The tissue sources of increased plasma bFGF in obese type 2 diabetes are not known. We have reported significant associations between increased plasma bFGF and waist-hip ratio or baseline thiazide diuretic use in the VADT (31). Thus one possibility is that weight gain and/or salt and water retention may increase post-baseline bFGF in part through enhanced expression of angiotensin II receptors (32). Whether increased plasma bFGF is a marker for counter-regulatory responses involving activation of the sympathetic nervous system or renin-angiotensin system requires more study.

A novel mechanism for long-lasting growth promotion in association with high plasma bFGF may involve growth stimulatory autoantibodies similar to those we described in cancer subjects which mimicking fibroblast growth factor in diverse tissues which express FGF receptors (33). In current VADT preliminary experiments, a 1:800 dilution of the IgG fractions (1-2 ug/mL) from sixteen VADT plasmas displayed increased mean endothelial cell growth promoting activity at increasing concentrations of plasma bFGF ranging from 0-10 (113%, n=7), 11-20 (120%, n=4) or >20 pg/mL (134%, n=5). Since post-baseline increased plasma bFGF may reflect persistent RAS activation, angiotensin II's known pro-immune effects (34) might promote an immune response to substantially increased plasma FGF in a subset of diabetes. Interestingly, the peak purified IgG endothelial cell activity in three (high bFGF) VADT plasmas bound and required high concentrations (0.25 or 0.4M) of Na phosphate to be eluted from a hydroxyapatite column, similar to the properties of cancer sera FGF-like autoantibodies in patients with osseous metastases (33). Diabetic plasma growth stimulatory autoantibodies had specific activity corresponding to 0.2 to 0.5 ng-eq bFGF/mL plasma which is 4-10 times higher than bFGF concentrations associated with a significantly increased risk for coronary heart disease occurrence in VADT substudy participants (7).

A limitation of our study is that it is small and may only reflect the experience of middle-aged and older obese men with treatment-resistant diabetes. More study is needed to confirm these findings in other populations with type 2 diabetes. It is possible that determination of post-baseline bFGF at year 1 may have underestimated the strength of a possible association between post-baseline bFGF and ongoing CVD risk in a subset of longstanding type 2 diabetes, since events occurring during the first year of follow-up were excluded from the analysis. More study is needed to test whether earlier post-baseline bFGF measurement may improve its sensitivity as a potential marker for CVD risk associated with treatment intensification used with longstanding diabetes.

Taken together, the present findings suggest that intensive glucose-lowering interacts differentially with diabetes duration to alter CVD risk in a subset of adult veterans with type 2 diabetes. In a high risk subset of adults with ten or fewer years of type 2 diabetes, aggressive glucose-lowering might reduce the risk for macrovascular as well as microvascular complications. The optimal treatment approach needed to avoid increasing CVD risk in adults with long-standing type 2 diabetes is still uncertain, however, and may require further clarification of the underlying pathophysiologic mechanisms.

TABLE 5
Baseline and follow up characteristics in the 399 study patients

| Variable | Baseline | Follow up* | p-value |
|---|---|---|---|
| Age | 59.3 ± 8.5 | | |
| Diabetes duration | 11.4 ± 7.6 | | |
| Male | 96% | | |
| Prior CV event | 37% | | |
| Hypertension | 70% | | |
| Race | | | |
| Non-Hispanic White | 57% | | |
| African-American | 21% | | |
| Hispanic | 18% | | |
| Current smoking | 18% | 12% | 0.0001 (n = 397) |
| bFGF (pg/mL) | 15.1 ± 25.8 (n = 399) | 8.0 ± 16.3\hat{ }(n = 215) | 0.0014 (n = 215) |
| HbA₁C (%) | 9.5 ± 1.4 | 8.0 ± 1.6 | <0.0001 (n = 396) |
| Systolic bp (mm Hg) | 130.9 ± 17.4 | 126.5 ± 16.6 | <0.0001 (n = 393) |
| BMI (kg/m²) | 30.9 ± 4.6 | 32.1 ± 5.5 | <0.0001 (n = 397) |
| Weight (lbs) | 212.7 ± 37.3 | 220.4 ± 42.0 | <0.0001 (n = 397) |
| Waist/hip ratio | 0.991 ± 0.07 | 0.997 ± 0.07 | 0.0661 (n = 362) |
| Total cholesterol (mg/dL) | 183.1 ± 43.9 | 159.6 ± 35.1 | <0.0001 (n = 377) |
| LDL cholesterol (mg/dL) | 107.8 ± 33.3 | 88.7 ± 30.2 | <0.0001 (n = 365) |
| HDL cholesterol (mg/dL) | 36.9 ± 9.7 | 39.5 ± 11.6 | <0.0001 (n = 377) |
| Triglycerides (mg/dL) | 195.3 ± 175.4 | 163.4 ± 104.7 | <0.0001 (n = 377) |
| Serum creatinine (mg/dL) | 0.99 ± 0.2 | 1.16 ± 0.36 | <0.0001 (n = 393) |

Results for continuous variables are displayed as mean +/− SD;
*year 3 or previous non-missing value prior to year 3;
\hat{ }year 1 annual visit.

TABLE 6
Cox proportional hazard regression; time to first post-baseline CHD event

| Variable | HR | 95% CI | p-value |
|---|---|---|---|
| Baseline bFGF | 1.012 | 1.006-1.019 | 0.0003 |
| Prior CV event | 3.488 | 2.182-5.575 | <0.0001 |
| Duration of | | | 0.964 |
| diabetes | | | |
| Intensive treatment | | | 0.0382 |
| Duration × treatment | | | 0.0190 |

n = 399 subjects;
HR—hazard ratio,
CI—confidence interval

TABLE 7
Cox proportional hazard regression: time to first post-baseline CVD event

| Variable | HR | 95% CI | p-value |
|---|---|---|---|
| Baseline bFGF | 1.008 | 1.002-1.014 | 0.0108 |
| Prior CV event | 3.498 | 3.079-3.807 | <0.0001 |
| AGE | 1.025 | 1.002-1.048 | 0.0330 |
| Duration of | | | 0.8868 |
| diabetes | | | |
| Intensive treatment | | | 0.0118 |
| Duration × treatment | | | 0.0289 |

n = 399 subjects;
HR—hazard ratio,
CI—confidence interval

### References for Example 2

1. The Diabetes Control and Complications Trial Research Group. The effect of intensive treatment of diabetes on the development and progression of long-term complications in insulin-dependent diabetes mellitus, New Engl J Med 1993; 329:683-689.
2. Control Group, Turnbull F M, Abraira C, Anderson R J, Byington R P, Chalmers J P, Duckworth W C, Evans G W, Gerstein H C, Holman R R, Moritz T E, Neal B C, Ninomiya T, Patel A A, Paul S K, Travert F, Woodward M. Intensive glucose control and macrovascular outcomes in type 2 diabetes. Diabetologia. 2009; 52(10:2288-98. Erratum in: Diabetologia. 2009; 52(1):2470.
3. The Accord Study Group. Effects of intensive glucose lowering in type 2 diabetes. N Engl J Med. 2008, 358: 2545-2559.
4. The ADVANCE Collaborating Group. Intensive blood glucose control and vascular outcomes in patients with type 2 diabetes. N Engl J Med 2008; 358:2560-2572.
5. Duckworth W, Abraira C, Moritz T, Reda D, Emanuele N, Reaven P D, Zieve F J, Marks J, Davis S N, Hayward R, Warren S R, Goldman S, McCarren M, Vitek M E, Henderson W G, Huang G D; VADT Investigators. Glucose control and vascular complications in veterans with type 2 diabetes, N Engl J Med. 2009; 360(2): 129-139. Erratum in: N Engl J Med. 2009; 361(10):1024-1025. N Engl J Med. 2009; 361(10):1028
6. Reaven P D, Moritz T E, Schwenke D C, Anderson R J, Criqui M, Detrario R, Emanuele N, Kayshap M, Marks J, Mudaliar S, Harsha Rao R, Shah J H, Goldman S, Reda D J, McCarren M, Abraira C, Duckworth W; Veterans Affairs Diabetes Trial. Intensive glucose-lowering therapy reduces cardiovascular disease events in veterans affairs diabetes trial participants with lower calcified coronary atherosclerosis. Diabetes, 2009; 58(11): 2642-2648.
7. Zimering M B, Anderson R J, Ge L, Moritz T E and VADT Investigators. Increased plasma basic fibroblast growth factor is associated with coronary heart disease in adult type 2 diabetes, Metabolism, Mar. 5, 2010, Epub.
8. Zimering M B, Eng J. Increased basic fibroblast growth factor-like substance in plasma from a subset of middle-aged or elderly male diabetic patients with microalbuminuria or proteinuria. J Clin. Endo Metab. 1996; 81: 4446-4452.
9. Hughes S E, Crossman D, Hall P A. Expression of basic and acidic fibroblast growth factors and their receptor in normal and atherosclerotic human arteries. Cardiovasc Res. 1993; 27(7): 1214-1219.
10. Flugelman M Y, Virmani R, Correa R, Yu Z X, Farb A, Leon M B, Elami A, Fu Y M, Casscells W, Epstein SE Smooth muscle cell abundance and fibroblast growth factors in coronary lesions of patients with nonfatal unstable angina. A clue to the mechanism of transformation from the stable to the unstable clinical state. Circulation 1993; 88(6): 2493-2500.
11. Schulz, M W, Chamberlain, C G, de Iongh, R U, McAvoy J W. Acidic and basic FGF in ocular media and lens: implications for lens polarity and growth patterns. Development 1993; 118(1): 117-126.
12. Hu F B, Hankinson S E, Stampfer M J, Manson J E, Colditz G A, Speizer F E, Hennekens C H, Willett W C. Prospective study of cataract extraction and risk of coronary heart disease in women. American Journal of Epidemiology, 2001; 153(9): 875-881.
13. Abraira C, Duckworth W, McCarren M, Emanuele N, Area D, Reda D, Henderson W. Design of the cooperative study of glycemic control and complications in diabetes mellitus type 2. J Diab & Compl 2003; 17: 314-322.
14. Zimering M B. Effect of intravenous bisphosphonates on release of basic fibroblast growth factor in serum of patients with cancer-associated hypercalcemia. Life Sciences 2002; 70: 1-14.
15. Larsson A, Skoldenberg E, Ericson H, Serum and plasma levels of FGF-2 and VEGF in healthy blood donors. Angiogenesis 2002; 5:107-110.
16. Schmieder R E, Hilgers K F, Schlaich M P, Schmidt B M. Renin-angiotensin system and cardiovascular risk. Lancet. 2007, 369(9568):1208-1219.
17. Zimering M B, Anderson R J, Moritz T E, Ge L and VADT Investigators. Low plasma basic fibroblast growth factor is associated with laser photocoagulation in adults from the Veterans Affairs Diabetes Trial, Metabolism, 58(3) 393-400, 2009.
18. Effect of intensive blood-glucose control with metformin on complications in overweight patients with type 2 diabetes (UKPDS 34). UK Prospective Diabetes Study (UKPDS) Group. Lancet. 1998; 352(9131):854-65.
19. Marso S P, Kennedy K F, House J A, McGuire D K. The effect of intensive glucose control on all-cause and cardiovascular mortality, myocardial infarction and stroke in persons with type 2 diabetes mellitus: a systematic review and meta-analysis Diabetes and Vascular Disease Research 2010 7(2): 119-130.
20. Gospodarowicz D, Ferrara N, Schweigerer L, Neufeld G, Structural characterization and biological functions of fibroblast growth factor. Endocr Rev. 1987; 8: 95-114.
21. Gajdusek C M, Carbon S. Injury-induced release of basic fibroblast growth factor from bovine aortic endothelium. J Cell Physiol 1989; 139: 570-579.
22. Sato Y, Hamanaka R, Ono J, Kuwano M, Rifkin D B, Takaki R. The stimulatory effect of PDGF on vascular smooth muscle cell migration is mediated by the induction of endogenous basic FGF. Biochem Biophys Res COMMA 1991; 174(3): 1260-1266.
23. Lindner V, Reidy M A. Proliferation of smooth muscle cells after vascular injury is inhibited by an antibody against basic fibroblast growth factor. Proc. Natl. Acad. Sci, USA 1991; 88: 3739-3743.
24. Esch F, Baird A, Ling N, Ueno N, Hill F, Denoroy L, Klepper R, Gospodarowicz D, Bohlen P, Guillemin R. Primary structure of bovine pituitary basic fibroblast growth factor (FGF) and comparison with the amino-terminal sequence of bovine brain acidic FGF. Proc Nat Acad Sci (USA). 1985; 19: 6507-6511.
25. Katinioti A A, Tousoulis D, Economou E, Stefanadis C, Trikas A, Tentolouris C, Pitsavos C, Androulakis A, Toutouzas P. Basic fibroblast growth factor changes in response to coronary angioplasty in patients with stable angina. Int J Cardiol. 2002; 84(2-3): 195-199.
26. Kraemer R, Pomerantz K B, Joseph-Silverstein J, Hajjar D P. Induction of basic fibroblast growth factor mRNA and protein synthesis in smooth muscle cells by cholesterol ester enrichment and 25-hydroxycholesterol. J Biol Chem. 1993; 268(11): 8040-8045.
27. Cucina A, Scavo M P, Muzzioli L, Coluccia P, Ceccarini S, Fuso A, Cavallaro A. High density lipoproteins downregulate basic fibroblast growth factor production and release in minimally oxidated-LDL treated smooth muscle cells. Atherosclerosis. 2006; 189(2): 303-309.
28. Gosgnach, W, Challah, M, Coulet, F, Michel, J-B, Battle, T. Shear stress induces angiotensin converting enzyme expression in cultured smooth muscle cells: possible involvement of bFGF. Cardiovasc Research 2000; 45(2): 486-492.
29. Kaneko T, Fujii S, Matsumoto A, Goto D, Ishimori N, Watano K, Furumoto T, Sugawara T, Sobel B E, Kitabatake A. Induction of plasminogen activator inhibitor-1 in endothelial cells by basic fibroblast growth factor and its modulation by fibric acid. Arterioscler Thromb Vast Biol. 2002; 22(5): 855-860.
30. Itoh H, Mukoyama, M, Pratt R, Gibbons G, Dzau V. Multiple autocrine growth factors modulate vascular smooth muscle cell growth response to angiotensin II. J. Clin Invest 1993; 91: 2268-2274.
31. Zimering M B, Anderson R J, Lou, P, Moritz T E. Investigators for the VADT. Plasma basic fibroblast growth factor is correlated with plasminogen activator inhibitor-1 concentration in adults from the Veterans Affairs Diabetes Trial. Metabolism. 2008; 57: 1563-1569.
32. Nickenig G, Strehlow K, Roeling J, Zolk O, Knorr A, Böhm M. Salt induces vascular AT₁ receptor overexpression in vitro and in vivo. Hypertension. 1998, 31: 1272-1277.
33. Zimering M B, Thakker-Varia S. Increased fibroblast growth factor-like autoantibodies in serum from a subset of patients with cancer-associated hypercalcemia. Life Sciences. 2002; 71(25): 2939-2959
34. Weidanz J A, Jacobson L M, Muehrer R J, Djamali A, Hullett D A, Sprague J, Chiriva-Internati M, Wittman V, Thekkumkara T J, Becker B N. ATR blockade reduces IFN-gamma production in lymphocytes in vivo and in vitro. Kidney Int. 2005 67(6):2134-42.

What is claimed is:

1. A method for diagnosing an increased risk of cardiovascular disease (CVD) in patients with type 2 diabetes comprising detecting basic fibroblast growth factor (bFGF) in a sample from the patient, an increased level of bFGF being indicative of increased risk of CHD, thereby diagnosing an increased risk of CHD in diabetic patients.

2. The method of claim 1, wherein the step of detecting comprises:
a. contacting the sample from the patient with an agent that binds basic fibroblast growth factor (bFGF) in the sample so as to form a complex; and
b. detecting the complex so formed thereby detecting the presence of bFGF in the sample.

3. The method of claim 2, wherein the step of detecting further comprises quantitatively determining the amount of bFGF in the sample.

4. The method of claim 2, wherein the agent is an antibody or fragment thereof that binds bFGF.

5. The method of claim 2, wherein the agent is labeled with a detectable marker.

6. The method of claim 1, wherein the step of detecting comprises:
a. contacting the sample with an antibody or fragment thereof that recognizes and binds bFGF and contacting the antibody or fragment thereof so bound with a second antibody or fragment thereof labeled with a detectable marker so as to form a complex; and
b. detecting the complex so formed, thereby detecting the presence of bFGF in the sample.

7. The method of claim 5 or 6, wherein the detectable marker is selected from the group consisting of a radiolabel, an enzyme, a chromophore and a fluorescer.

8. The method of claim 1, wherein the sample is a biological fluid.

9. The method of claim 8, wherein the biological fluid is urine, blood serum or plasma.

10. A method for monitoring the course of a pathological complication in a subject with type 2 diabetes which comprises quantitatively determining in a first biological sample from the subject the presence of basic fibroblast growth factor (bFGF) by the method of claim 1, and comparing the amount so determined with the amount present in a second biological sample from the subject, such samples being taken at different points in time, a difference in the amounts of basic fibroblast growth factor (bFGF) determined being indicative of the course of the pathological condition.

11. The method of claim 10, wherein the pathological complication is cardiovascular disease (CVD).

12. The method of claim 1, wherein CVD is selected from the group consisting of coronary heart disease (CHD), myocardial infarction, coronary revascularization, inoperable coronary artery disease and cardiovascular death.

13. The method of claim 1, wherein type 2 diabetes is advanced type 2 diabetes.

14. A method for diagnosing an increased risk of CVD in patients with type 2 diabetes comprising detecting basic fibroblast growth factor (bFGF) in a sample from the patient, the level of bFGF is selected from the group consisting of
a. greater than or equal to 50 pg/ml;
b. greater than or equal to 45 pg/ml;
c. greater than or equal to 40 pg/ml;
d. greater than or equal to 35 pg/ml;
e. greater than or equal to 30 pg/ml;
f. greater than or equal to 25 pg/ml; and
g. greater than or equal to 20 pg/ml;
the level of bFGF of any of a-g being indicative of increased risk of CVD, thereby diagnosing an increased risk of CVD in diabetic patients.

15. The method of claim 14, wherein the level of bFGF is between 20 pg/ml to 60 pg/ml.

16. A method for treating CVD in patients with type 2 diabetes comprising administering to the subject an effective amount of an antibody or fragment thereof that recognizes and binds basic fibroblast growth factor (bFGF) in a subject, thereby treating CVD.

17. The method of claim 4 or 16, wherein the antibody is selected from the group consisting of a polyclonal antibody, a monoclonal antibody, a chimeric antibody, a humanized antibody and a fully-human antibody.

18. The method of claim 4 or 16, wherein the antibody fragment is selected from the group consisting of Fv fragments, F(ab′) fragments, single chain antibodies and F(ab′)₂ fragments.

19. The method of claim 14 or 16, wherein CVD is selected from the group consisting of coronary heart disease (CHD), myocardial infarction, coronary revascularization, inoperable coronary artery disease and cardiovascular death.
